(19) United States
(12) Patent Application Publication
(10) Pub. No.: US 2022/0088259 A1
(43) Pub. Date: Mar. 24, 2022

(54) Device For Disinfecting Air With Electromagnetic Radiation

(71) Applicant: ebm-papst Mulfingen GmbH & Co. KG, Mulfingen (DE)

(72) Inventors: Ralph Wystup, Kunzelsau (DE); Markus Humm, Weissbach (DE); Rainer Nase, Weikersheim (DE); Frederik Wystup, Neuenstein (DE)

(21) Appl. No.: 17/469,832

(22) Filed: Sep. 8, 2021

(30) Foreign Application Priority Data
Sep. 23, 2020 (DE) 10 2020 124 739.7

(51) Int. Cl. A61L 9/18; A61L 9/03

(52) U.S. Cl. CPC A61L 9/18; A61L 2209/134; A61L 9/032

(57) ABSTRACT

A device (1) for disinfecting air with electromagnetic radiation, has a radiation chamber (10) where air flows from an intake side (A) to a discharge side (B) along a flow path. At least one radiation source (11) generates electromagnetic radiation in the microwave range and emits electromagnetic radiation into the radiation chamber. At least one fan (20) with an impeller (21) generates an air flow through the radiation chamber (10). The fan (20) takes in air on the suction side (A), convey it through the radiation chamber (10) along the flow path to the discharge side (B) and blows it out of the radiation chamber (10) on the discharge side (B). At least the impeller (21) is arranged inside the radiation chamber (10). The impeller (21) has a plurality of blades (22) formed at least in sections from a material deflecting the electromagnetic radiation.

### CROSS-REFERENCE TO RELATED APPLICATION

This application claims priority to German Patent Application No. 10 2020 124 739.7 filed Sep. 23, 2020. The entire disclosure of the above application is incorporated herein by reference.

### FIELD

The disclosure relates to a device for disinfecting air with electromagnetic radiation in the microwave range.

### BACKGROUND

Some bacteria and viruses, such as Covid-19, may be present in air as aerosols or attached to or enveloped by water droplets. Accordingly, it is desirable to be able to purify as large as possible quantities of air from these bacteria and viruses, particularly when using air conditioning, or generally in enclosed spaces.

Various approaches for disinfecting air are already known in the prior art. However, most of them are not suitable for continuously disinfecting large quantities of air, that is, for rendering the bacteria or viruses present in the air harmless.

For example, it is already known to disinfect the air by UVC light. In addition, disinfecting air by microwave radiation or heat is also known in principle. However, the devices provided for this purpose, in the prior art, usually only permit the cleaning of comparatively small quantities of air.

The underlying problem of the disclosure is therefore to overcome the aforementioned disadvantages. The present disclosure provides a device and an associated method where the largest possible quantities of air can be effectively and efficiently disinfected or purified.

### SUMMARY

This problem is solved by a device for disinfecting air with electromagnetic radiation including a radiation chamber with air flow along a flow path from an intake side to a discharge side. At least one radiation source is configured to generate electromagnetic radiation in a microwave range and to emit electromagnetic radiation into the radiation chamber. A frequency of the electromagnetic radiation is selected so that water molecules present in the air are excited to an oscillation that heats the water molecules. The air flowing through the radiation chamber is exposed to the electromagnetic radiation. The water present in the air is brought to a temperature of at least 100° C. The device further comprises at least one fan with an impeller for generating an air flow through the radiation chamber. The fan is configured to take in air on the intake side, to convey it through the radiation chamber along the flow path to the discharge side. It blows the air it out of the radiation chamber on the discharge side. At least the impeller is arranged in the radiation chamber. The impeller has a plurality of blades that are formed at least in sections from a material that deflects or reflects the electromagnetic radiation.

According to the disclosure, a device is proposed for disinfecting air with electromagnetic radiation in the microwave range. The device has a radiation chamber where air flows along a flow path from an intake side to a discharge side. Furthermore, the device has at least one radiation source that is configured to generate electromagnetic radiation in the microwave range and to emit the electromagnetic radiation into the radiation chamber. A frequency of the electromagnetic radiation is selected so that water molecules present in the air are excited to an oscillation. This heats the water molecules. Thus, the air flowing through the radiation chamber is exposed to the electromagnetic radiation and water present in the air is brought or heated to a temperature of at least 100° C. The air flowing through the radiation chamber is disinfected by exposing the air to electromagnetic radiation or microwave radiation.

As described in the introduction, the viruses and bacteria present in the air, which are also referred to as particles below, are usually present as aerosols or in water droplets adhering to the air. Thus, water or water molecules are always present. Most viruses and also most bacteria can be killed in boiling water. Thus, only the water in the air must be heated to such an extent. Thus the particles are killed or rendered harmless in the process. The proposed device or associated method involves heating the water in the air by electromagnetic radiation in the microwave range, by microwave radiation.

Microwaves or their electric field component heat materials with a dipole moment, such as water molecules, by setting the molecules in torsional vibration. The frequency of the microwaves or the radiation is only decisive insofar as it must not be too high. Thus, the molecules can still follow the rotary motion of the alternating field.

The penetration depth calculated as a result of the skin effect is in the cm range in water (1 cm-5 cm), depending on the temperature of the water. As the frequency of the waves or radiation increases, the penetration depth decreases, but the energy conversion increases as a result of the shorter wavelength. The penetration depth is defined as the depth where the initial intensity of the waves has dropped to 37%. This means that the waves also reach the areas below the penetration depth, but the heating there is significantly lower than above that depth. The optimum frequency would therefore be one whose associated penetration depth corresponds to the mean radius of the particle distribution in the air around 5 μm to 0.5 mm. However, this is not economically feasible due to the very high frequency of over 30 GHz required for this purpose, since there are hardly any suitable radiation sources. Likewise, at this high frequency, the water molecules would no longer perform sufficient rotational motion as described.

Therefore, an absorption frequency of approx. 2.45 GHz that deviates from a resonance frequency and is therefore not optimal, is sufficient for heating water. The frequency of the electromagnetic radiation does not have to be exactly 2.45 GHz, but it is advantageous if the frequency is in a range around 2.45 GHz and, for example, between 2.00 and 3.00 GHz. Magnetrons can be used as a radiation source for generating such electromagnetic radiation in the range of 2.45 GHz, and preferably exactly 2.45 GHz. They are also used in commercial microwave ovens. Thus they can be provided extremely cost-efficiently.

The use of microwaves or electromagnetic radiation instead of other variants known in the prior art, such as disinfecting air by UVC light, is useful because a high throughput of air can be achieved.

Unlike UVC light, microwave radiation itself cannot break organic compounds due to its low energy. This is possible with microwaves purely through heating as a result of the dipole oscillations. So the heating is the same as if you would heat the compound on the stove.

For this purpose, sufficient energy must be introduced into the particles in a short time, since they preferably only stay or should stay in the radiation chamber for a short time.

Since commercially available magnetrons can contribute a good 500 watts of RF power at about 60% efficiency, and multiple magnetrons or radiation sources can be used, very high radiation power can be achieved using multiple radiation sources in the radiation chamber.

To increase efficiency, the apparatus according to the disclosure further comprises at least one fan with an impeller generating an air flow through the radiation chamber. The fan is designed to take in air or, more precisely, ambient air at the intake side. It conveys it through the radiation chamber along the flow path to the discharge side and blow it out of the radiation chamber on the discharge side. At least the impeller is arranged in the radiation chamber. The impeller has a plurality of blades that are formed at least in sections from a material that deflects or reflects the electromagnetic radiation. The fan blades or the impeller thus acts as a reflector known from commercial microwave ovens. Thus, the beams emitted by the at least one radiation source are reflected. The reflection avoids standing waves inside the radiation chamber. The standing waves would otherwise cause static hot and cold zones inside the radiation chamber. Thus, in addition to the air conveyance along the flow path, the fan or impeller is a reflector used for field homogenization.

The fan can be arranged completely in the radiation chamber. In this case, a motor, rotationally driving the impeller, is preferably shielded from the electromagnetic radiation in the radiation chamber. Alternatively, the motor can be located outside the radiation chamber and drive the impeller in the radiation chamber by a drive shaft leading into the radiation chamber from the outside. If a fan or at least an impeller is provided on both the intake and discharge sides, the impellers can also be connected and driven via a common drive shaft.

A sufficiently large quantity of air or a sufficiently large volume flow can be disinfected, an air flow through the radiation chamber generated by the fan, the length of the radiation chamber through which air flows, and the number of radiation sources are adjusted to one another. Thus, the particles contained in the selected quantity of air or the particles contained in the volume flow can be exposed to a sufficiently large radiation energy. Accordingly, the desired quantity of air can flow through the radiation chamber, within a predetermined time, can be substantially completely disinfected in the process.

Preferably, the radiation chamber is formed of an electromagnetic radiation attenuating or shielding and/or reflecting material. Thus, no electromagnetic radiation can escape from the radiation chamber.

A shielding element is arranged to prevent microwave radiation from escaping from the radiation chamber on the intake side or the discharge side. The shielding element, through which air can flow, is arranged on the intake side and the discharge side of the radiation chamber. The element is made of a material that attenuates or shields and/or reflects the electromagnetic radiation. Preferably, the intake and discharge sides are closed with a metal grid. The mesh size is selected according to the wavelength or frequency of the electromagnetic radiation.

In a variant, the at least one impeller may further integrally form the screening element on the intake side or act as an additional screening element on the intake side. Alternatively or additionally, the at least one impeller or another impeller may integrally form the shielding element on the discharge side.

According to another advantageous variant, the shielding element, arranged on the discharge side of the radiation chamber, is configured as a flow obstacle. The air pressure in the radiation chamber is increased to increase the residence time of the air or the particles present in the air in the radiation chamber and the duration of irradiation. For this purpose, the shielding element can, for example, simply reduce the flow cross-section or the area through which air can flow. The use of a shielding element as a flow obstacle also leads to an increase in pressure in the radiation chamber and, consequently, at the same time to a higher particle density that increases the efficiency of disinfection.

In a likewise advantageous further development, the radiation chamber has, at least in sections, a circular flow cross section through which air can flow. The at least one impeller has a diameter corresponding to the flow cross section. In this context, corresponding means that the impeller directly adjoins a wall of the radiation chamber defining the flow cross-section towards the radial outside. The diameter of the circular flow cross-section is only slightly larger than the outer diameter of the impeller. Thus, rotation of the blade wheel is possible without hindrance.

In another variant, the device, particularly in the case of air that is very dry on the intake side, also comprises at least one moistening or humidifying device. The device is arranged along the flow path upstream of the radiation chamber or on the intake side at least in sections inside the radiation chamber. This ensures that all viruses and/or bacteria or all particles of the air drawn into the radiation chamber on the intake side are enveloped in water or at least adhere to water. The moistening device is configured to humidify the air flowing through the radiation chamber before it is exposed to the electromagnetic beams.

For example, the at least one moistening device may be an ultrasonic water atomizer.

The use of a moistening device is also advantageous in combination with a fan arranged on the intake side of the radiation chamber or on the intake side in the radiation chamber. Thus, the humidified air is swirled again by the fan and mixed before the air is exposed to radiation.

Another embodiment of the device is particularly advantageous when using a moistening device, but also generally when air that is as dry as possible or air with a predetermined air humidity is to be discharged on the discharge side. In this embodiment the device has at least one dehumidification device that is arranged along the flow path downstream of the radiation chamber or at least in sections inside the radiation chamber on the discharge side. The dehumidification device is configured to dehumidify the air flowing through the radiation chamber after it has been exposed to the electromagnetic radiation. Thus, after decontamination, water is removed from the humid air.

The at least one dehumidification device may be at least one, preferably plate-shaped, condenser, for example, a thermal condenser. Such a condenser can also be configured as one or more metal plates. The condenser removes water from the air by condensation of the air on the plates.

In conjunction with a condenser, a water outlet is also provided on the discharge side. The water separated from the air at the condenser can be discharged through the outlet from the device as condensate.

In another embodiment the device has an air supply duct arranged along the flow path on the intake side of the radiation chamber and encloses the radiation chamber at least in sections. Further, it is advantageous if the at least one radiation source and also the at least one moistening device are arranged at least with a heat sink on an outer wall of the radiation chamber or on the outer side of the radiation chamber. Thus, at least some of the intake air flows along the outside of the radiation chamber and preferably cools it. If the at least one radiation source and/or the at least one moistening device are provided at least with their heat sink on the outside of the radiation chamber, they are advantageously located inside the air supply duct. Thus, air drawn in along the heat sink or heat sinks absorbs heat, is preheated, and at the same time cools the components before the air is drawn into the radiation chamber on the intake side.

If the device is combined with other equipment, such as an air conditioning system, the air stream drawn into the radiation chamber on the intake side may also be a cooling air stream. It is used along the flow path upstream of the radiation chamber to cool components of the other equipment.

At least one pre-filter can be provided along or in the flow path on the intake side of the radiation chamber. Foreign bodies can be filtered out of the air through the pre-filter. In this context, foreign bodies means for example dust or other foreign bodies that are larger than the particles that are to be rendered harmless by the irradiation in the radiation chamber and would have a disturbing effect in the radiation chamber.

Another aspect of the disclosure further relates to a method for air disinfection by microwaves with a device according to the disclosure. Air is taken in on the intake side by the fan and conveyed along the flow path through the radiation chamber to the discharge side. The air is exposed to the electromagnetic radiation generated by the at least one radiation source along the flow path in the radiation chamber. Thus, water contained in the air is brought to a temperature of at least 100° C. Accordingly, bacteria or viruses surrounded by the water or adhering to it are rendered harmless.

All features disclosed above can be combined in any desired manner, where technically feasible and not contradictory.

Other advantageous further developed embodiments of the disclosure are disclosed in the dependent claims and/or are described in more detail through the drawings in conjunction with the description of the preferred embodiment of the disclosure.

### DRAWINGS

FIG. 1 is a sectional view of a device for disinfecting air with electromagnetic radiation.

### DETAILED DESCRIPTION

The FIGURE is an exemplary schematic and shows a device 1 for disinfecting air, that is substantially formed by three sections or assemblies. The radiation chamber 10 forms the central section or a first assembly. Outside or ambient air is drawn into the radiation chamber 10 through an upstream air supply duct 40, as a second assembly. After the air has been exposed to electromagnetic radiation or, more precisely, to microwaves in the radiation chamber 10, to disinfect the air, it is directed or blown into a downstream third fluidic assembly. In this case, it is used to dehumidify the air by a dehumidification device 30.

In or adjacent to the radiation chamber 10, two magnetrons are provided as radiation sources 11. Each magnetron emits electromagnetic radiation in the microwave range at a frequency of 2.45 GHz into the radiation chamber 10. This is indicated by the arrows in the radiation chamber 10. Radiation is reflected by the outer walls of the radiation chamber 10 by the shielding elements 13 and by the impeller 21 or its blades 22. Thus, the surroundings are shielded from the electromagnetic radiation.

The power of the radiation sources and the flow of air through the radiation chamber from its intake side A to its discharge side B are coordinated so that the water contained in the air is substantially completely boiled. Thus, contained particles, i.e. viruses and bacteria, are substantially completely neutralized. For this purpose the following can be taken into account: the length of the flow path, the velocity of the flow of air through the radiation chamber 10, the air pressure in the radiation chamber 10, or the differential pressure in the radiation chamber 10 with respect to an environment of the device 1, and also, for example, the humidity of the air flowing into the radiation chamber 10 on the intake side.

In order to assume sufficient water or sufficient humidity of the irradiated air, particularly in the case of dry air on the intake side, two moistening devices 12 are provided. The moistening devices 12 are in the form of ultrasonic water atomizers in the variant shown. The devices moisten the air flowing into the radiation chamber 10 on an intake side A. In this case, it is also particularly advantageous that the fan 20 is provided in the flow direction downstream of the moistening devices 12. The fan swirls the moistened air and thus increases the mixing of the air with moisture.

Advantageously, moreover, the blades 22 of the impeller 21 of the fan 20 are formed, at least in sections, of a material reflecting the electromagnetic radiation, such as metal. Thus, the impeller 21 integrally serves as a rotating reflector through which the electromagnetic beams are chaotically deflected in the radiation chamber 10. The chaotic reflection prevents standing waves within the radiation chamber 10. Thus, hot and cold zones do not occur and the air flowing through the chamber is heated uniformly or the water contained therein is brought to a complete boil.

The fan 20 has a motor 23 for driving its impeller 21. In the embodiment shown the motor 23 is arranged in the radiation chamber 10. Alternatively, it may be arranged outside. The motor 23 is preferably arranged on a side of the impeller 21 facing away from the radiation sources 11. It is shielded from the electromagnetic radiation since the impeller 21 acts as a shielding element. Additionally the motor 23 may have its own shielding and be shielded from electromagnetic radiation.

Depending on how extensive the shielding effect is provided by the impeller 21, the shielding element 13 arranged on the intake side A can be eliminated, since its function is then integrally taken over by the impeller 21.

Two magnetrons are provided as radiation sources 11. They are shown opposite each other. Depending on the radiation power to be introduced and also depending on the volumetric flow to be disinfected, a plurality of preferably uniformly distributed radiation sources 11 can be provided in the circumferential direction, particularly in the case of a radiation chamber 10 with a circular cross-section. Additionally, these may also be arranged adjacent to each other along the flow path and distributed along the length of the radiation chamber 10.

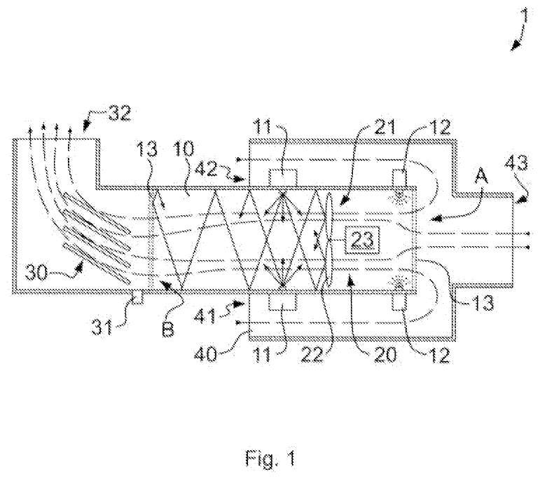

In the variant of the device 1 shown in FIG. 1, the radiation sources 11 and the moisture penetration devices 12 or at least cooling elements of these components are arranged on the outside of the outer wall of the radiation chamber 10. The air supply duct 40 surrounds the radiation chamber 10 in the section where these components are arranged. Thus, at least part of the air flow drawn by the fan 20 from the surroundings of the device 1 into the radiation chamber 10 passes along these components in a cooling manner.

The air flow is shown in the FIGURE as dashed arrows. The air flows is drawn in through the inlet openings 41, 42 to cool the radiation sources 11 and the moistening devices 12 that are preheated at the same time. Thus, the air flowing into the radiation chamber 10 or the water contained in the inflowing air can be brought to a boil more quickly in the radiation chamber 10. Although the inflowing air can also be used completely for cooling components arranged outside the radiation chamber 10, that air is additionally drawn in through another inflow opening 43, in the present case.

Incidentally, the air supply duct 40 can also completely annularly surround the radiation chamber 10. Thus, the two inflow openings 41, 42 form an annular inlet or a single annular inflow opening.

The air flowing through the device 1, shown in a dashed line, is blown out of the radiation chamber 10 on the discharge side B into a dehumidification device 30. In the present case it is configured as four thermal condensers arranged adjacent to one another. The water present in the air or the humid, water vapor-laden air is dehumidified by this dehumidifying device 30. Thus, the air can flow out at the outlet 32 of the device 1 is at a predetermined humidity. The water condensing on the condensers can be discharged from the device 1 via a drain opening 31.

Execution of the disclosure is not limited to the preferred exemplary embodiments mentioned above. Instead, a number of variants are conceivable that make use of the solution presented, even with fundamentally different designs.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A device for disinfecting air with electromagnetic radiation, comprising:
a radiation chamber with air flow along a flow path from an intake side to a discharge side; and
at least one radiation source configured to generate electromagnetic radiation in a microwave range and to emit electromagnetic radiation into the radiation chamber, a frequency of the electromagnetic radiation is selected so that water molecules present in the air are excited to an oscillation that heats the water molecules, and the air flowing through the radiation chamber is exposed to the electromagnetic radiation and water present in the air is brought to a temperature of at least 100° C.;
the device further comprises at least one fan with an impeller for generating an air flow through the radiation chamber, the fan is configured to take in air on the intake side, to convey it through the radiation chamber along the flow path to the discharge side and to blow it out of the radiation chamber on the discharge side;
at least the impeller is arranged in the radiation chamber and the impeller has a plurality of blades that are formed at least in sections from a material that deflects or reflects the electromagnetic radiation.

2. The device according to claim 1, wherein
a shielding element, through which air can flow, is arranged on the intake side and the discharge side of the radiation chamber, the shielding element is formed from a material that attenuates and/or reflects the electromagnetic radiation.

3. The device according to claim 2, wherein the at least one impeller integrally forms the shielding element on the intake side.

4. The device according to claim 2, wherein the shielding element arranged on the discharge side of the radiation chamber is configured as a flow obstacle where the air pressure in the radiation chamber is increased.

5. The device according to claim 1, wherein the radiation chamber has a circular flow cross-section, and the at least one impeller has a diameter corresponding to the flow cross-section.

6. The device according to claim 1, further comprising at least one moistening device arranged along the flow path in front of the radiation chamber or on the intake side at least in sections inside the radiation chamber and the moistening device configured to moisten the air flowing through the radiation chamber before it is exposed to electromagnetic beams.

7. The device according to the claim 6, wherein the at least one moistening device is an ultrasonic water atomizer.

8. The device according to claim 1, further comprising at least one dehumidifying device arranged along the flow path in front of the radiation chamber or on the intake side at least in sections inside the radiation chamber and the dehumidifying device configured to dehumidify the air flowing through the radiation chamber after it has been exposed to electromagnetic beams.

9. The device according to claim 8, wherein the at least one dehumidifying device is a condenser.

10. The device according to claim 1, further comprising an air supply duct arranged along the flow path on the intake side of the radiation chamber the air supply duct encloses the radiation chamber at least in sections, such that at least part of the intake air flows along the outside of the radiation chamber.

11. A method for air disinfection by microwaves with a device according to claim 1, wherein air is taken in on the intake side by the fan and conveyed along the flow path through the radiation chamber to the discharge side and is exposed to the electromagnetic radiation along the flow path in the radiation chamber, such that water contained in the air is brought to a temperature of at least 100° C.
